(19) United States
(12) Patent Application Publication
(10) Pub. No.: US 2005/0042701 A1
(43) Pub. Date: Feb. 24, 2005

(54) Anti-petasin antibodies, methods for the production thereof and their use

(76) Inventors: Werner Schobler, Schwanebeck (DE); Christian Hentschel, Birkholz (DE); Vivianne Tack, Frankfurt am Main (DE)

Correspondence Address: GOODWIN PROCTER, 103 EISENHOWER PARKWAY, ROSELAND, NJ, 07068, US

(21) Appl. No.: 09/844,290

(22) Filed: Apr. 27, 2001

Related U.S. Application Data
(63) Continuation of application No. PCT/DE99/03525, filed on Nov. 1, 1999.

(30) Foreign Application Priority Data
Oct. 30, 1998 (DE) 198 50 011.4
Nov. 30, 1998 (DE) 198 56 777.4

(51) Int. Cl. G01N 33/53; G01N 33/537; G01N 33/543; C12P 21/04; C07K 16/18

(52) U.S. Cl. 435/7.92; 530/387.1; 435/70.21

(57) ABSTRACT

The invention relates to an anti-petasin antibody for detecting petasin or petasin protein conjugates in physiological fluids, the antibody being free of cross reactivity to derivatives, structural analogs or metabolites of petasin, method for producing the antibody, and a process for detcting petasin or petasin protein conjugates in physiological fluids.

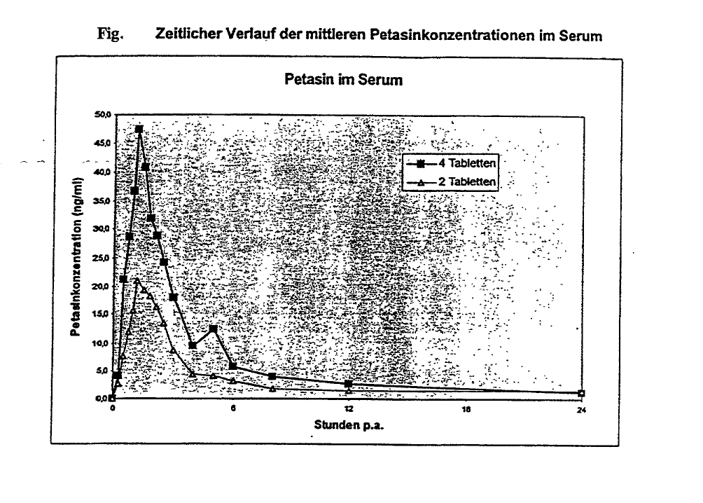

### FIELD OF INVENTION

The invention relates to anti-petasin antibodies for detecting petasin or petasin protein conjugates in physiological liquids which do not show any cross reactivity to derivatives, structural analogs or metabolics of petasin, methods for producing them by immunization by petasin derivatives which are suitably coupled to a carrier molecule, and to their use and a test kit.

### BACKGROUND

Petasin, a component of butterbur extracts is a known ester of petasol and angelic acid which already for a longer time has been used as vegetable spasmoanalgesic for combatting spasms of the gastrointestinal tract, in particular ureteral colics, spastic bronchititis and migraine and also as an antiphlogistic (B. Debrunner et al.; Pharm. Acta Helv. 72, 359-380 (1998). In addition, an antitumor effect is attributed to petasin drugs (B. Meier et al., Hagers Handbuch der pharmazeutischen Praxis (Manual of pharmaceutical practice), 5^{th} edition, p. 81-105, Springer-Verlag (1994)). Also the latest findings relating to the effects on the biosynthesis of leukotrienes are available (D. Pichl et al., Planta Medica, 60, 318-322 (1994)).

After oral application of petasin drugs only concentrations in the range of a few ng/ml are to be expected in body fluids of healthy subjects. Due to this background biological, physical and chemical methods of detection applied in characterizing the drug itself cannot be used for quantifying petasin in body fluids. Even most up-to-date analytical methods such as the HPLC usually applied are not sufficiently sensitive or not suitable due to their large time requirement for large numbers of samples.

### BRIEF DESCRIPTIONS OF THE INVENTION

It is for that reason that the object of the present invention is to provide methods of detecting petasin, in particular suitable methods with a high sensitivity and specificity which allow a good bioavailability for the desired pharmacokinetic investigations.

The immunochemical methods of detection according to the present invention meet the requirements for sensitivity and specificity thus not requiring an additional extraction or concentration of the sample to advance the proper determination as it is required when applying chromatographic methods of the prior art.

It was possible to accomplish this task by providing an anti-petasin antibody for detecting petasin or petasin protein conjugates in physiological fluids wherein the antibody does not show any cross reactivity to derivatives, structural analogs or metabolites of petasin.

The antibodies according to the present invention are produced by preparing polyclonal or monoclonal antibodies by mammals and/or birds with petasin or a derivative thereof, which are suitably coupled to a carrier molecule. It was found, to our surprise, that thus it was possible to avoid a production of antibodies directed against the coupling group of petasin or a potentially occurring modification of the immunodominant epitope situated in the vicinity of position 8.

### BRIEF DESCRIPTION OF THE DRAWING

The invention is described in greater detail below, with reference being had to the sole figure of the drawing, showing the mean petasin concentration changes of petasin in serum, as a function of time.

### DETAILED DESCRIPTION

The polyclonal or monoclonal antibodies are produced by immunization of mammals and/or birds by petasin or petasin derivatives of the Formula (I) 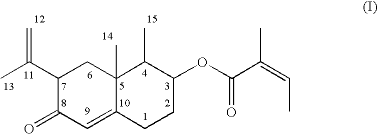
and obtained by hybridoma techniques or recombinantly with the aid of antibody libraries.

The following derivatives coupled to a carrier molecule are suitably used:
(a) Derivatives of petasin of Formula I where the keto group in position 8 is replaced by a carboxyl group and coupled to a bovine serum albumin by EDAC;
(b) Derivatives of petasin of Formula I where the keto group in position 8 is replaced by a carboxyl group and coupled to a bovine serum albumin or fibrinogen through an activated hydrazide dextran with the carboxyl group being suitably inserted with carboxymethylhydroxyamine forming oxime;
(c) Derivatives of petasin of Formula I where the double bond in positions 11, 12 is brominated and coupled to bovine serum albumin activated by means of a Traut's reagent; and
(d) Derivatives of petasin of Formula I where angelic acid has been split off and the remaining petasol has been coupled to a carrier through chloroformic acid ester.

The anti-petasin antibodies thus produced do not show any crossreactivity to derivatives, structural analogs or metabolites of petasin and are used for detecting petasin or petasin-protein conjugates in physiological liquids with either petasin, petasin protein conjugates or the anti-petasin antibodies suitably showing a marker, such as enzymes, fluorescent dyes, radioisotopes or redoxactive compounds. The reactants are suitably available in a homogeneous solution.

Petasin bound to antibodies is optically, electrochemically, fluorimetrically or radiochemically detected, suitably optically by means of color reagents or by chromatography.

In one embodiment of the present invention either anti-petasin antibodies, the petasin to be detected, or the petasin protein conjugates are bound onto a solid phase with a washing process taking place between the reaction steps.

The solid phase is suitably chemically activated, wherein adsorptive or covalent bonding takes place of the anti-petasin antibodies, or the petasin to be determined, or the petasin-protein conjugates. Polystyrene is suitably used as solid phase.

In addition, the solid phase can have a differing geometric shape, thus e.g. the shape of a microtitration plate, a tube or have a spherical or plane shape.

The invention furthermore relates to a test kit for detecting petasin in physiological liquids comprising anti-petasin antibodies, a solid phase, such as polystyrene, washing solution, dilution buffer, marked petasin or a marked anti-species antibody, a marker-specific detection system, suitably an enzyme substrate.

The invention is hereinafter explained in greater detail by reference to the following examples.

### EXAMPLES

A) Production of Immunogenes

Petasin Oxime:

10 mg (3.3×10⁻⁵ mol) of petasin are dissolved in 5 ml of ethanol, 15 mg (6.8×10⁻⁵ mol) of carboxymethoxylamine hemihydrochloride (Sigma-Aldrich) are added and 5 M sodium hydroxide solution are added drop by drop until a pH of 12 is reached. The batch is refluxed for 4 hours, evaporated to dryness on a water bath, washed with 2 M hydrochloric acid and dissolved in a mixture of 1 ml of dioxane and 2 ml of DMSO and stored at −70° C. Thin-layer chromatography: R_{f} value (silica gel G60, chloroform)=0.42 (petasin:0.16).

Oxime is formed as sole reaction product.

Petasin oxime bovine serum albumin:

32 mg (4.8×10⁻⁷ mol) of bovine serum albumin (BSA) are to be dissolved in 4 ml of PBS (solution A). 7 mg (1.8×10⁻⁵ mol) of petasin oxime, dissolved in 1 ml of dioxane/DMSO=1:2 (v/v), 16 mg of 1-ethyl-3-(3-dimethylaminopropyl) carbodiimide (EDAC) are added and while stirring incubated for 30 min. at room temperature (solution B). Solution B is added dropwise to solution A, stirred for 6 hours at room temperature, subsequently dialyzed at 4° C. against 3×0.5 l of PBS (0.01 M phosphate, 0.15 M NaCl, pH=7.4) and stored at −70° C.

Petasin-Dextran Proteins:

7 mg (1.8×10⁻⁵ mol) of petasin oxime, dissolved in 1 ml of dioxan/DMSO=1:2 (v/v) are added dropwise to 32 mg of bovine serum albumin (4.8×10⁻⁷ mol) or fibrinogen in 4 ml of PBS, and 0.5 mg (1.5×10⁻⁴ mol hydazide groups) of activated hydrazide dextran (Pierce, Code 20900) are added. Thereupon, 16 mg of 1-ethyl-3-(3-dimethylaminopropyl(carbodiimide (EDAC) are added and the mixture is incubated for 4 hours at room temperature. Thereupon, a dialysis is carried out at 4° C. against 3×0.5 l of PBS (0.01 M phosphate, 0.15 M NaCl, pH=7.4). Storage is at −70° C.

Bromopetasin Bovine Serum Albumin:

Brominating of Petasin:

10 mg (3.1×10⁵ mol) of bromine in 1 ml of dichloromethane, dissolved in 3 ml of dichloromethane, are added drop by drop with swirling to 5 mg of (3.3×10⁻⁵ mol) petasin. Thereupon, the batch is evaporated to dryness on a water bath and taken up in 1 ml of DMSO.

Thin-layer chromatography: R_{f} value (silica gel G60, chloroform)=0.51 (petasin:0.16).

Thiolation of Bovine Serum Albumin:

40 mg (6×10⁻⁷ mol) of bovine serum albumin is dissolved in 1 ml 0.1 M of phosphate buffer, pH=8.0, and 20 mg (1.4×10⁴ mol) of 2-iminothiolane hydrochloride (Traut's reagent) are added and incubated for 40 min. at room temperature. Subsequently, with the aid of a column filled with Sephadex G25 (1×10 cm) a buffer exchange is carried out against 0.1 M phosphate buffer at pH=7.2. 4 mg (8.4×10⁻⁶ mol) of bromopetasin are added while stirring for dissolving the thiolated protein and an incubation for 3 hours at room temperature, thereupon dialysis is carried out at 4° C. against 3×0.5 l of PBS (0.01 M phosphate, 0.15 M NaCl, pH=7.4).

Petasol Bovine Serum Albumin:

0.7 mg (2.9×10⁻⁶ mol) of petasol are dissolved in 200 μl of dried dioxane/DMF=1:1 (v/v), 2 mg (7.9×10⁻⁶ mol) of 5-norbornene-2,3-dicarboximidyl chloroformic acid ester and 4 mg (3.3×10⁻⁵ mol) of 4-dimethyl amino pyridine are added and the mixture is incubated for 1 hour at room temperature excluding atmospheric humidity. Thereupon, this solution is added dropwise with stirring to 10 mg (1.5×10⁻⁷ mol) of bovine serum albumin, dissolved in 0.5 ml of PBS and incubated for 2 hours at room temperature. Thereupon, it is dialyzed at 4° C. against 3×0.5 l of PBS (0.01 M phosphate, 0.15 M NaCl, pH=7.4) and protein conjugate is stored at −70° C.

B) Production of Anti-Serum

Immunization is administered in rabbits as primary subcutaneous and intramuscular with always 3 mg of petasin-BSA in a complete Freund's adjuvant. The secondary injection is effected four weeks after the primary injection. After further two weeks the first booster injection is administered, a second one is carried out twelve weeks after the beginning of immunization, in an incomplete Freund's adjuvant. About eight weeks after starting immunization a first blood sample is taken which is supplemented by a further one after four weeks. Exsanguination is carried out after 16 weeks.

The antisera obtained are subjected to a titer determination for specific anti-petasin antibodies by an enzyme immunoassay where petasin ovalbumin is bound to the surface of microtitration plates. The antisera to be examined and the normal sera of the rabbits are subsequently incubated in a dilution series with the immobilized petasin. The bound antibodies are detected by incubation with a goat-anti-rabbit immunoglobuline enzyme conjugate (peroxidase) and subsequent visually evaluable substrate reaction.

C) Enzyme Immunoassay

Petasin Ovalbumin:

4 mg of EDAC are added to 0.3 mg (8×10⁻⁷ mol) of petasin oxime, dissolved in 100 μl of dioxane/DMSO=1:2 (v/v) and incubated for 30. min. at room temperature. Subsequently, the batch is put into a solution of 5.5 mg (1.2×10⁻⁷ mol) of ovalbumin in 3 ml of PBS, incubated for 2 hours at room temperature while being stirred and subsequently for 16 hours at 4° C. The reaction mixture is dialysed at 4° C. against 3×0.5 l of aqua bidest. and the protein conjugate is stored at −70° C.

Coating:

Petasin ovalbumin is adsorptively bound to polystyrene microtitration plates in a concentration of 5 mg/P in 0.1 M carbonate buffer, pH=9.5, (100 μl/well) for 16 hours at 4° C. and thereupon sucked off. After washing it two times with 300 μl/well washing buffer (PBS, 0.1% Tween 20) it is blocked for 2 hours at room temperature with 150 μl/well blocking solution (0.6% gelatine, 0.02% sodium acid in PBS) and subsequently washed three times with washing buffer.

Execution of the Test:

50 μl of the serum sample to be tested or the respective standard (1:4 dilution in a sample buffer (PBS, 1% BSA, 0.1% Tween 20, 0.01% thiomersal) and 50 μL of an optimized anti-serum dilution in a sample buffer are simultaneously incubated with shaking for 1 hour at room temperature. Subsequently, the microtitration plate is washed three times with 300 μl/well of washing buffer and incubated for 30 min. at room temperature with 100 μl of anti-rabbit immunoglobulin peroxidase conjugate, diluted in sample buffer, and once more washed as above. Thereupon, it is incubated for 10 min. with 100 μl of a substrate solution ready for use (3.3′, 5.5′-tetramethyl benzidine) per well and the reaction is stopped by adding 100 μl/well of 0.5 M sulfuric acid. The evaluation is carried out at 450 nm in a microtitration plate reader.

Description of Indication

Plant extracts obtained by special methods from leaves or rhizomes of Petasites hybridus L. can inhibit the 5-lipoxygenase. Thus, the arachidonic acid cascade is effectively interrupted in the case of allergic inflammations. In particular, the release of leukotriene from endogenic cells stimulated in the case of inflammations is stopped, inter alia also from eosinophilic and neutrophilic leukocytes.

Thus, such plant extracts are potential candidates for the therapeutic use in the case allergic inflammations such as allergic rhinitis, asthma, atopic dermatitis, colitis ulcerosa etc. First clinical experience proves the therapeutic efficiency of this plant extract in the case of allergic rhinitis. A prophylactic use of the extract in the case of selected forms of migraine also gave indications to its efficiency.

In addition to detecting the plasma level required for the efficiency for relevant components of the extract, e.g. petasin, the knowledge of the pharmacokinetics of such relevant components is urgently required for a medical use of the plant extract. With the anti-petasin antibodies according to the present invention in an enzyme immunoassay a secure detection of petasin in the blood in the lower ng range is achieved. The results of the following pharmacokinetic examination proves impressively its usability.

In a 1^{st} phase of the clinical test for determining the pharmacokinetic parameters of tablets containing butterbur extract was a single oral administrations of 2 or 4 tablets to 24 clinically healthy men at the age between 18 and 40 years.

The open, crossover test was chosen as method, single administration of each dose in a randomized order with an interval of at least 7 days between the administrations.

Efficiency:

Model-independent pharmacokinetic parameters for petasin Statistical methods:

ANOVA, ANOVA_{log}, Wilcoxon-Mann-Whitney test, Wilcoxon-sign order test

### SUMMARY—CONCLUSIONS

Results:

Petasin serum concentration (ng/ml)

| after administering 2 tablets | | | | | | | after administering 4 tablets | | | | | |
|---|---|---|---|---|---|---|---|---|---|---|---|---|
| Time | | | | | | | | | | | | |
| p.a.(h) | N | Mean | S.D. | Min. | Median | Max. | N | Mean | S.D. | Min. | Median | Max. |
| 0 | 0 | 0.0 | | 0.0 | | 0.0 | 0 | 0.0 | | 0.0 | | 0.0 |
| 0.25 | 9 | 2.8 | 1.8 | 1.1 | 2.2 | 5.5 | 13 | 4.2 | 3.7 | 1.0 | 3.2 | 14.9 |
| 0.5 | 20 | 7.6 | 5.8 | 1.5 | 5.9 | 23.3 | 21 | 21.2 | 24.9 | 1.3 | 13.7 | 96.2 |
| 0.75 | 20 | 11.9 | 5.7 | 4.0 | 11.0 | 23.8 | 19 | 28.7 | 22.5 | 2.5 | 23.0 | 91.8 |
| 1 | 20 | 15.6 | 7.2 | 4.0 | 14.7 | 29.3 | 20 | 36.6 | 23.0 | 7.8 | 38.1 | 100.0 |
| 1.17 | 20 | 21.0 | 16.1 | 5.6 | 15.3 | 62.9 | 20 | 47.3 | 29.1 | 7.5 | 43.6 | 100.0 |
| 1.5 | 20 | 19.3 | 12.0 | 5.1 | 16.1 | 47.3 | 19 | 40.8 | 22.3 | 12.2 | 32.4 | 90.7 |
| 1.833 | 20 | 18.2 | 11.3 | 7.8 | 14.7 | 44.3 | 20 | 32.0 | 20.1 | 13.8 | 26.8 | 100.0 |
| 2.167 | 20 | 16.3 | 8.3 | 7.3 | 14.5 | 31.7 | 20 | 28.9 | 15.0 | 11.4 | 27.5 | 76.1 |
| 2.5 | 20 | 13.6 | 6.4 | 5.9 | 10.2 | 26.6 | 19 | 24.3 | 10.7 | 8.4 | 26.1 | 40.9 |
| 3 | 20 | 8.8 | 4.1 | 3.1 | 7.7 | 18.3 | 20 | 17.9 | 10.0 | 7.2 | 14.6 | 49 |
| 4 | 20 | 4.5 | 2.6 | 1.7 | 5.2 | 11.2 | 20 | 9.5 | 5.2 | 2.9 | 8.1 | 20.8 |
| 5 | 20 | 4.1 | 2.3 | 1.5 | 3.4 | 8.8 | 21 | 12.4 | 16.5 | 3.2 | 7.3 | 81.4 |
| 6 | 18 | 3.2 | 1.7 | 1.2 | 3.1 | 8.1 | 21 | 5.8 | 3.8 | 1.6 | 5.0 | 14.7 |
| 8 | 18 | 1.9 | 0.8 | 1.0 | 1.6 | 4.2 | 19 | 3.9 | 3.1 | 1.4 | 3.1 | 15.7 |
| 8 | 18 | 1.9 | 0.8 | 1.0 | 1.6 | 4.2 | 19 | 3.9 | 3.1 | 1.4 | 3.1 | 15.7 |
| 12 | 13 | 1.6 | 0.5 | 1.1 | 1.4 | 2.9 | 18 | 2.7 | 1.0 | 1.3 | 2.5 | 5.1 |
| 24 | 6 | 1.5 | 0.5 | 1.1 | 1.3 | 2.3 | 10 | 1.3 | 0.4 | 1.0 | 1.0 | 2.3 |

Values below the detection limit (1 ng/ml) are equated with 0.

Values below the detection limit (1 ng/ml) are equated with 0.

Model-independent pharmacokinetic parameters (±S.D.)

| parameter/dosage | 2 tablets | 4 tablets |
|---|---|---|
| Cₘₐₓ (ng/ml) SD | 25.5 ± 14.8 | 58.1 ± 26.7 |
| tₘₐₓ (h) SD | 1.616 ± 0.499 | 1.614 ± 0.926 |
| AUC₀₋ₜ₍ₗₐₛₜ₎ (ng/ml * h) SD | 65.30 ± 35.61 | 151.15 ± 68.21 |
| AUC₀₋ᵢ (ng/ml * h) SD | 79.68 ± 42.27 | 168.22 ± 73.43 |
| AUC_{Rest} (%) SD | 18.3 ± 7.9 | 10.8 ± 4.9 |
| T_{1/2} (h) SD | 7.155 ± 4.611 | 7.618 ± 3.338 |
| MRT (h) SD | 7.32 ± 3.74 | 6.74 ± 2.47 |

Security Parameters:
No significant and clinically relevant modifications of the haematological and clinical.chemical laboratory parameters
Undesired Events:
Undesired events did not occur.
Conclusions:
The resorption takes quickly place depending on the dose.
Both dosages shall be regarded to be equal as to their bioavailability.
   Mathematical-Statistical Evaluation
1. Pharmacokinetic and statistical calculations
   The serum levels of petasin measured were the basis of the evaluation.
2. Model-independent pharmacokinetic parameters

The averages and standard deviations (SD) of the pharmacokinetic parameters are shown in Table 1.

| Parameter/dosage | 2 tablets | 4 tablets |
|---|---|---|
| Cₘₐₓ (ng/ml) ± SD | 25.5 ± 14.8 | 58.1 ± 26.7 |
| tₘₐₓ (h) ± SD | 1.616 ± 0.499 | 1.614 ± 0.926 |
| AUC₀₋ₜ₍ₗₐₛₜ₎ (ng/ml * h) ± SD | 65.30 ± 35.61 | 151.15 ± 68.21 |
| AUC₀₋ᵢ (ng/ml * h) ± SD | 79.68 ± 42.27 | 168.22 ± 73.43 |
| AUC_{Rest} (%) ± SD | 18.3 ± 7.9 | 10.8 ± 4.9 |
| t_{1/2} (h) ± SD | 7.155 ± 4.611 | 7.618 ± 3.338 |
| MRT (h) ± SD | 7.32 ± 3.74 | 6.74 ± 2.47 |

The dose-dependent parameters Cₘₐₓ and AUC are nearly proportional to the dose, the deviations of the averages of all other parameters are nearly identical considering the standard deviations that were determined.

The big standard deviations have to be regarded as an expression of interindividual differences, notably of the speed of resorption, distribution and metabolism of petasin. Thus, after administering the low dose a petasin serum level above the determination limit of the analyzing method has not been detected at no time.

The calculation of the relevant bioavailability (calculation of the dose-corrected quotient of the pharmacokinetic parameters with a 90% confidence interval) of the dose of 4 tablets compared with a dose of 2 tablets of the test medication shows:

TABLE 2
Comparative bioavailability
Butterbur 4 tablets versus butterbur 2 tablets

| | | Cₘₐₓ | T/R (%) | AUC₀₋ᵢ | T/R (%) |
|---|---|---|---|---|---|
| adoption of | Statistical | point | Conf. interv. | Point | Conf. interv. |
| distributions | method | estimation | from . . . to | estimation. | from . . . tos |
| normal distr | ANOVA (x-Over) | 113.5 | 91.9 . . . 135.0 | 106.2 | 86.5 . . . 126.0 |
| log-normal | ANOVA | 114.9 | 94.7 . . . 139.5 | 109.1 | 92.5 . . . 128.6 |
| distrib. | log (x-Over) | | | | |
| distribution- | Wilcoxon-Mann- | 113.5 | 87.7 . . . 141.8 | 101.0 | 87.5 . . . 121.3 |
| free | Whitney Test | | | | |
| | Wilcoxons sign-order-test | 111.4 | 93.4 . . . 135.4 | 104.5 | 90.8 . . . 122.3 |

In the framework of the limits between 70 and 142.9% for Cₘₐₓ and between 80 and 125% for AUC usually accepted in bioavailability tests the availability of both dosages is to be regarded as equal.

TABLE 3
Groups stastistic of the petasin concentration (ng/ml) in
serum after administering 4 tablets

| Time | | | | | | |
|---|---|---|---|---|---|---|
| p.a. | N | Mean | S.D. | Min. | Median | Max. |
| 0 | 0 | <1 | | | <1 | |
| 0.25 | 13 | 4.2 | 3.7 | 1.0 | 3.2 | 14.9 |
| 0.5 | 21 | 21.2 | 24.9 | 1.3 | 13.7 | 96.2 |
| 0.75 | 19 | 28.7 | 22.5 | 2.5 | 23.0 | 91.8 |
| 1 | 20 | 36.6 | 23.0 | 7.8 | 38.1 | 100.0 |
| 1.167 | 20 | 47.3 | 29.1 | 7.5 | 43.6 | 100.0 |
| 1.5 | 19 | 40.8 | 22.3 | 12.2 | 32.4 | 90.7 |
| 1.833 | 20 | 32.0 | 20.1 | 13.8 | 26.8 | 100.0 |
| 2.167 | 20 | 28.9 | 15.0 | 11.4 | 27.5 | 76.1 |
| 2.5 | 19 | 24.3 | 10.7 | 8.4 | 26.1 | 40.9 |
| 3 | 20 | 17.9 | 10.0 | 7.2 | 14.6 | 49.0 |
| 4 | 20 | 9.5 | 5.2 | 2.9 | 8.1 | 20.8 |
| 5 | 21 | 12.4 | 16.5 | 3.2 | 7.3 | 81.4 |
| 6 | 21 | 5.8 | 3.8 | 1.6 | 5.0 | 14.7 |
| 8 | 19 | 3.9 | 3.1 | 1.4 | 3.1 | 15.7 |
| 12 | 18 | 2.7 | 1.0 | 1.3 | 2.5 | 5.1 |
| 24 | 10 | 1.3 | 0.4 | 1.0 | 1.0 | 2.3 |

Values below the detection limit (1 ng/ml) correspond to 0.

From the attached Figure there can be seen that the medium maximum petasin concentration (Cₘₐₓ) has nearly doubled after administering double the dose. The medium time of reaching the maximum serum level (tₘₐₓ) remains constant.

1-22. (canceled)

23. Anti-petasin antibody for detecting petasin or petasin protein conjugates in physiological fluids, said antibody being free of cross reactivity to derivatives, structural analogs or metabolites of petasin.

24. A method of producing anti-petasin antibodies which comprises producing polyclonal or monoclonal antibodies by immunization of mammals and/or birds with petasin or petasin derivatives of Formula I 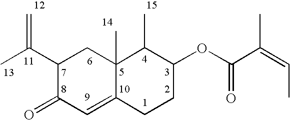
and antibodies are obtained by means of the hybridoma technique or recombinantly with the aid of antibody libraries.

25. The method of claim 24 wherein derivatives coupled to carrier molecules are used as petasin derivatives for immunization.

26. The method of claim 25 wherein derivatives of petasin are used for immunization where the keto group in position 8 of Formula I. was replaced by a carboxyl group and coupled to bovine serum albumin by means of EDAC.

27. The method of claim 25 wherein derivatives of petasin are used for immunization where the keto group in position 8 of Formula I. was replaced by a carboxyl group and coupled to a bovine serum albumin through activated hydrazide dextran or fibrogen.

28. The method of claim 26 wherein the insertion of carboxyl group is carried out with carboxymethylhydroxyamine forming oxime.

29. The method of claim 27wherein the insertion of carboxyl group is carried out with carboxymethylhydroxyamine forming oxime.

30. The method of claim 25wherein derivatives of petasin are used for immunization where the double bond in positions 11, 12 of Formula I. is bromated and coupled to bovine serum albumin by means of a Traut's reagent.

31. The method of claim 25wherein derivatives of petasin are used for immunization where angelic acid is split off and the remaining petasol is coupled to a carrier through chloroformic acid ester.

32. A process for detecting petasin or petasin in protein conjugates in physiological fluids, by anti-petasin antibodies.

33. The process of claim 32, wherein said anti-petasin antibodies do not show any cross reactivity to derivatives, structural analoges or metabolites of petasin.

34. The process of claim according to claim 32 wherein petasin, petasin protein conjugates, or anti-petasin antibodies are equipped with a marker.

35. The process of claim 34 wherein said marker is an enzyme, fluorescent dye, radio isotope, or a redoxactive compound.

36. The process of claim 32, which comprises detecting petasin bound to antibodies by optical, electrochemical, fluorimetrical or radiochemical technique.

37. The process of claim 36, wherein a color reagent is used for detecting petasin bound to antibodies.

38. The process of claim 36, wherein said detecting is carried out chromatographically.

39. The process of claim 32, wherein reactants are present in a homologous solution.

40. The process of claim 32, wherein anti-petasin antibodies, the petasin to be detected, or the petasin protein conjugates are bound to a solid phase and washing is carried out between reaction steps.

41. The process of claim 40 wherein anti-petasin antibodies, the petasin to be detected, or the petasin protein conjugates is adsorptively or covalently bound to a solid phase after chemical activation of the solid phase.

42. The process of claim 40 wherein the solid phase is polystyrene.

43. The process of claim 40 wherein the solid phase has a differing geometric shape.

44. The process of claim 43 wherein said geometric shape is that of a microtitration plate, a tube, in a spherical or plane shape.

45. A test kit for detecting petasin in physiological fluids which comprises anti-petasin antibodies, a solid phase, a washing solution, a dilution buffer, and enzyme marked petasin.
